(19) United States
(12) Patent Application Publication
(10) Pub. No.: US 2005/0266085 A1
(43) Pub. Date: Dec. 1, 2005

(54) Gelled emulsion and microemulsion formulations for dermal drug delivery

(76) Inventors: Kevin S. Warner, West Jordan, UT (US); Jie Zhang, Salt Lake City, UT (US)

Correspondence Address: THORPE NORTH & WESTERN, LLP., 8180 SOUTH 700 EAST, SUITE 200, P.O. BOX 1219, SANDY, UT, 84070, US

(21) Appl. No.: 10/856,745

(22) Filed: May 28, 2004

(51) Int. Cl. A61K 9/70; A61K 9/14

(52) U.S. Cl. 424/486; 424/449

(57) ABSTRACT

The present invention is drawn to gelled emulsion and microemulsions formulations for dermal drug delivery, including transdermal drug delivery. In one embodiment, a drug-containing gelled emulsion can comprise a continuous gelled aqueous phase, and a discontinuous drug-containing oil phase dispersed within the continuous gelled aqueous phase, wherein the drug-containing gelled emulsion is present in a dermal delivery system. In another embodiment, a drug-containing microemulsion can comprise a continuous aqueous phase, a discontinuous oil phase including a lipophilic drug, and surfactant(s) substantially positioned interfacially between the continuous aqueous phase and the discontinuous oil phase. The discontinuous oil phase can be dispersed in the continuous aqueous phase, and the drug-containing microemulsion can be present in a dermal reservoir patch delivery system.

### FIELD OF THE INVENTION

The present invention is drawn to dermal drug delivery systems. More particularly, the present invention is drawn to gelled emulsion and microemulsion formulations for dermal drug delivery.

### BACKGROUND OF THE INVENTION

Dermal delivery of drugs and other active agents by the use of a transdermal drug delivery device, e.g., patch, is common for many different drug types, including water soluble drugs. Particularly with respect to transdermal delivery of drugs, the quantity of drug that permeates across the skin per unit area per unit time, or “flux,” is a significant parameter in determining whether a drug can be effectively delivered transdermally for a specific treatment regimen. Often, heat, electrical current, chemical permeation enhancers, or the like are used to facilitate the delivery such drugs into or through the skin. However, even with these techniques, there are many drugs that are difficult to deliver transdermally in an effective amount over an effective period of time to be desirable for use. Exemplary of such drugs that are often difficult to deliver transdermally include lipophilic drugs. Further, when such drugs are included in formulations that are effective from a delivery standpoint, other drawbacks related to the mechanics of ongoing administration can be a barrier to desirable use.

As such, it would be desirable to provide formulations, methods, and systems for delivering drugs dermally and transdermally, particularly lipophilic drugs that can be difficult to deliver across the skin. It would also be desirable to provide systems that can be effective for dosing lipophilic drugs, and also that will not be vulnerable to be wiped from the skin by external objects, such as clothing.

### SUMMARY OF THE INVENTION

It has been recognized that certain lipophilic drugs can be dermally delivered to subjects using certain gelled emulsion and/or microemulsion drug delivery systems. In accordance with this recognition, a drug-containing gelled emulsion can comprise a continuous gelled aqueous phase, and a discontinuous drug-containing oil phase dispersed within the continuous gelled aqueous phase, wherein the drug-containing gelled emulsion is present in a dermal delivery system. The drug-containing oil phase can comprise a pharmaceutically active lipophilic drug and an oil, wherein the lipophilic drug is at least five times more soluble in the oil than in water.

In another embodiment, a method of preparing a drug-containing gelled emulsion for dermal delivery can comprise steps of forming a drug-containing oil phase including a lipophilic drug and an oil, wherein the lipophilic drug is at least five times more soluble in the oil than in water; and forming an aqueous phase. A further step of emulsifying the oil phase with the aqueous phase to form an oil-dispersed emulsion can also be carried out, as well as a step of gelling the aqueous phase after the oil-dispersed emulsion is formed to form the drug-containing gelled emulsion. Additionally, a step of incorporating the drug-containing gelled emulsion in a dermal delivery system can also be carried out. The incorporating step can occur after the drug-containing gelled emulsion is formed, or the gel can be formed in the drug delivery system.

In a related embodiment, a drug-containing gelled microemulsion can comprise a continuous aqueous phase, and a discontinuous oil phase including a lipophilic drug and an oil, wherein the lipophilic drug is at least five times more soluble in the oil than in water. The discontinuous oil phase can be dispersed in the continuous aqueous phase and surfactant(s) can also be present that are substantially positioned interfacially between the continuous aqueous phase and the discontinuous oil phase to form an oil-in-water microemulsion. In one embodiment, the continuous aqueous phase can be gelled, though this is not required.

In another embodiment, a method of preparing a drug-containing microemulsion for dermal delivery can comprise steps of forming a drug-containing oil phase comprising a lipophilic drug and an oil, wherein the lipophilic drug is at least five times more soluble in the oil than in water; and forming an aqueous phase. Other steps include emulsifying the aqueous phase with the oil phase in the presence of at least one surfactant to form the drug-containing microemulsion, wherein the surfactant(s) are substantially positioned interfacially between a continuous aqueous phase and a dispersed discontinuous oil phase; and incorporating the drug-containing microemulsion in a dermal delivery system.

In a more detailed embodiment, a gel patch for dermal drug delivery can comprise an impermeable backing film, a drug-containing gelled emulsion or gelled microemulsion, and an adhesive. The drug-containing gelled emulsion can be in contact with the backing film and also be configured to directly contact a skin surface of a subject. The drug-containing gelled emulsion can include a continuous gelled aqueous phase and a discontinuous drug-containing oil phase dispersed within the continuous gelled aqueous phase. The drug-containing oil phase can comprise a pharmaceutically active lipophilic drug and an oil, wherein the lipophilic drug is at least five times more soluble in the oil than in water. The adhesive can be on the backing film and positioned peripherally with respect to the drug-containing gelled emulsion. The adhesive can further be configured to adhere the backing film to the skin surface, thus substantially sealing the drug-containing gelled emulsion between the skin surface and the backing film.

Additional features and advantages of the invention will be apparent from the following detailed description which illustrates, by way of example, features of the invention.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

As used in this specification and the appended claims, the singular forms “a,” “an,” and “the” include plural referents unless the content clearly dictates otherwise.

The term “dermal delivery” includes any method wherein formulations in accordance with embodiments of the present invention are delivered to, into, and through the skin of a subject, including topical delivery for skin treatment or absorption, or transdermal delivery for regional tissue or systemic administration. Thus, when referring to “dermal delivery” or “dermal delivery systems,” it is meant to include topical and/or transdermal delivery. Additionally, when applicable and allowed by the context of the specification and claims, instances of the phrase “dermal delivery” and “dermal delivery system” can be replaced with “transdermal delivery” and “transdermal delivery system” to describe more specific embodiments of the present invention.

The term “reservoir patch” refers to a dermal delivery system that typically includes four layers, though four layers are not strictly required. The four layers include an impermeable backing film which gives mechanical support; a liquid compartment containing a drug solution, gel, or suspension; a semi-permeable membrane; and an adhesive layer that contacts and adheres to the skin surface. In one embodiment, the semi-permeable membrane can also be the adhesive layer, thus being a three layer system. In accordance with embodiments of the present invention, the drug solution, gel, or suspension can be in the form of a microemulsion.

In contrast, a “single-layer drug-in-adhesive patch” which is a type of matrix patch, includes the drug directly within a skin-contacting adhesive. The adhesive in this formulation can serve two functions: first, to affix the system to the skin, and second, to serve as a foundation containing drug and any other ingredients or excipients under a backing film.

Another type of patch is a “semisolid patch” or “gel patch.” This type of patch includes an aqueous semisolid phase or hydrogel that contains an oily drug suspension. The drug-containing semisolid phase or hydrogel/oily drug suspension is typically in direct contact with the skin. A skin adhesion component can either be incorporated into the drug suspension or hydrogel itself, or can be present in a concentric or perimeter configuration around the drug-containing semisolid phase or hydrogel.

The term “lipophilic drug” can be defined as drugs that have low solubility in water, but which have much higher solubility in certain other liquids or oils, especially those liquids or oils that are not substantially soluble in water. Similarly, the term “oil” includes solvents or liquids that are substantially not soluble in water.

The term “microemulsion” can be defined as a system of water, oil, and surfactants, which typically are clear or otherwise transparent, and which are thermodynamically stable liquid. Typically, a microemulsion is transparent because the oil droplets are smaller than the wavelengths of visible light, e.g., from about 400 nm to 800 nm. In one embodiment, the microemulsion can be gelled and included in a drug delivery matrix patch, reservoir patch, or gel patch. If not gelled, the microemulsion can be included in a reservoir patch. Alternatively, the gelled microemulsions and non-gelled microemulsions can be applied topically as a lotion, ointment, or cream.

The term “emulsion” can be defined as a system including a continuous phase and a discontinuous phase. Typically, dispersed droplets (discontinuous phase) can be present in another liquid (continuous phase). An emulsifying agent may or may not also be present. The consistency of an emulsified system may range form a relatively low viscosity system, e.g., lotions, to more semisolid systems, e.g., creams. These emulsions can be included in a drug delivery matrix patch, reservoir patch, or gel patch. Alternatively, gelled emulsions can also be applied topically as a lotion, ointment, or cream.

The term “flux” can be defined as the quantity of drug that permeates across the skin per unit area per unit time. There are many drugs that fail to produce satisfactory fluxes in transdermal delivery devices. To illustrate the concept of flux, polyisobutylene (PIB) glue is a common component used in transdermal delivery matrices patches for transdermal drug delivery, and PIB glue based patches produce satisfactory transdermal fluxes for many drugs. One reason for such poor transdermal fluxes of some drugs can be due to their low solubility in typical solvent systems or matrices. It has been found that many lipophilic drugs, such as benzodiazepines, steroids, local anesthetics, antibiotics, and retinoids, have extremely low solubility in water and in some PIB based glues, which at least partially explains why water-based and PIB glue-based formulations produce such low fluxes for these and other similarly water insoluble drugs.

To illustrate, one can consider alprazolam. Through experimentation, it has been found that alprazolam has at least 5 times higher solubility than water in the following liquids: eugenol (clove oil), rose oil, n-methyl-pyrrolidone, isopropyl myristate, ethanol, oleyl alcohol, citronella oil, isopropyl alcohol, Labrasol, wintergreen oil, octyldodecanol, ethyl oleate, evening primrose oil, and orange oil. Further, the following liquids provided at least 20 times higher solubility than water: wintergreen oil, octyldodecanol, oleyl alcohol, ethanol, citronella oil, rose oil, eugenol, n-methyl pyrrolidone, isopropyl alcohol. Still further, the following liquids provided greater than 100 times higher solubility than water: ethanol, citronella oil, rose oil, n-methyl pyrrolidone, and isopropyl alcohol. It is to be emphasized that the above list of solubilizing agents is specific to alprazolam. As such, this list is applicable to this particular drug. This being stated, still, some of the solubilizing agents listed as having favorable solubilizing properties may work well with other lipophilic drugs, in accordance with embodiments of the present invention, as would be easily ascertainable to one skilled in the art. Though it is useful to know what compositions can be used to solubilize these and other similarly soluble medications for use in dermal delivery devices, applying a liquid formulation directly on the skin can be impractical in some devices, such as dermal delivery devices. This is because variable drug delivery quantities typically occur which can be caused by poorly defined contact area with the skin. Further, liquid formulations are vulnerable to be wiped from the skin by external objects, such as clothing.

One solution to this problem is to solubilize an active ingredient or medication in a solubilizing liquid (oil), such as one of those describe above or other oil that can solublize a given drug at least five times greater than water, and then include the solubilized drug in the form of a gel for dermal delivery. However, the liquids listed and other possible drug solubilizing liquids are not always capable of being gelled. Another approach would be to incorporate a liquid formulation, including the active ingredient solubilized in the solvent (oil), into a reservoir patch configuration. However, many oils are not compatible with known adhesive layers, rendering this approach difficult for practical application.

Thus, in accordance with embodiments of the present invention, certain formulations have been discovered that provide acceptable flux, as well as solve the problems that can occur when using more traditional dermal delivery devices.

Gelled Emulsion and Microemulsion Formulations

Formulations that have been discovered to be effective for providing dosing of lipophilic drugs by dermal delivery include the use of emulsions. In a first embodiment, a liquid that can solubilize lipophilic drugs can be selected for use. The liquid generally includes or is an oil that is immiscible in water. The lipophilic drug can be at least partially dissolved in the oil to form an oil phase. A water-based solution can also be prepared that includes at least one gelling agent that can be used to form a gel of the aqueous phase. The oil phase and the aqueous phase can then be emulsified. Appropriate emulsifying agent(s) can be used if desired. Once in an emulsified stage, the aqueous phase can then be gelled using a composition interactive with the gelling agent.

More specifically, a drug-containing gelled emulsion can comprise a continuous gelled aqueous phase, and a discontinuous drug-containing oil phase dispersed within the continuous gelled aqueous phase, wherein the drug-containing gelled emulsion is present in a dermal delivery system. The drug-containing oil phase can comprise a pharmaceutically active lipophilic drug and an oil, wherein the lipophilic drug is at least five times more soluble in the oil than in water.

In another embodiment, a method of preparing a drug-containing gelled emulsion for dermal delivery can comprise steps of forming a drug-containing oil phase including a lipophilic drug and an oil, wherein the lipophilic drug is at least five times more soluble in the oil than in water; and forming an aqueous phase. A further step of emulsifying the oil phase with the aqueous phase to form an oil-dispersed emulsion can also be carried out, as well as a step of gelling the aqueous phase after the oil-dispersed emulsion is formed to form the drug-containing gelled emulsion. Additionally, a step of incorporating the drug-containing gelled emulsion in a dermal delivery system can also be carried out. The incorporating step can occur after the drug-containing gelled emulsion is formed, or the gel can be formed in the drug delivery system.

The continuous gelled aqueous phase can include water and a gel-forming component, such as a gel-forming polymer, e.g., polyvinyl alcohol. Additionally, in some embodiments, the aqueous phase can include a gel triggering agent, reactive for the formation of a gel with the gel-forming component, e.g., boric acid or a salt of boric acid when reacting with polyvinyl alcohol.

In an alternative gelling embodiment, the continuous gelled aqueous phase can include a thermal gel that is flowable when heated above its melting point. In this state, the discontinuous drug-containing oil phase can be dispersed in the thermal gel above the melting point. Upon cooling, the drug-containing gelled emulsion is formed as the thermal gel reverts to below its melting point. Examples of such thermal gels include those having one or more gel-forming agent selected from the group consisting of carrageenan, pectin, and gelatin.

Turning to a related embodiment, a drug-containing gelled microemulsion can comprise a continuous aqueous phase, and a discontinuous oil phase including a lipophilic drug and an oil, wherein the lipophilic drug is at least five times more soluble in the oil than in water. The discontinuous oil phase can be dispersed in the continuous aqueous phase, where at least one surfactant is present initially either in the oil or aqueous phase, to form an oil-in-water microemulsion. Surfactants are substantially positioned interfacially between the continuous aqueous phase and the discontinuous oil phase. In one embodiment, the continuous aqueous phase can be gelled, though this is not required.

In another embodiment, a method of preparing a drug-containing microemulsion for dermal delivery can comprise steps of forming a drug-containing oil phase comprising a lipophilic drug and an oil, wherein the lipophilic drug is at least five times more soluble in the oil than in water; and forming an aqueous phase. Other steps include emulsifying the aqueous phase with the oil phase in the presence of at least one surfactant to form the drug-containing microemulsion, wherein the surfactant(s) is substantially positioned interfacially between a continuous aqueous phase and a dispersed discontinuous oil phase; and incorporating the drug-containing microemulsion in a dermal delivery system.

Though the microemulsions described above do not specifically require that they be gelled, it can be preferred that even the microemulsions be gelled as well. Exemplary gelling techniques that can be used include the use of a polyvinyl alcohol gelling component along with a boric acid gel triggering agent, as describe above. Alternatively, a thermal gel can also be used, as previously described.

In the microemulsion embodiments, the continuous aqueous phase can include water and at least one surfactant, such as fatty alcohols, mono- and diglycerides, and mixtures thereof. Alternatively, one or more surfactants may be selected from short chain alcohols, plurol isostearique, Tweens, Span 20, Chemophor RH, soybean lecithin, Labrasol, fatty alcohols, monoglycerides, dilycerides, and mixtures thereof. More generally, surfactant(s) can be selected from the group consisting of nonionic surfactants and zwitterionic surfactants. The presence of surfactants in the formulation can cause the composition to appear as a clear solution, though light scattering data would indicate that the composition is actually a fine dispersion, having discontinuous oil phase including aggregates with an average size less than about 400 nm.

To provide exemplary, non-limiting amounts of each component that can be present in the microemulsions, the weight amount continuous aqueous phase can be from 5 wt % to 95 wt %, the amount of the surfactant(s) can be from 0.1 wt % to 95 wt %, and the amount of the oil phase can be from 0.1 wt % to 30 wt %.

With respect to both the gelled emulsion embodiments and the microemulsion embodiments described above, the lipophilic drug can be at least twenty times more soluble in the oil than in water, and in another embodiment, at least one hundred times more soluble in the oil than in water. Solubility can be determined by experimentation or by referring to reference materials that provide relevant information.

The dermal delivery system can be a dermal delivery patch, such as a reservoir patch, a gel patch, or a matrix patch. Typically, the drug delivery system used can be a gel patch. However, if an adhesive can be incorporated in the drug-containing gelled emulsion, the drug delivery system can be an adhesive-containing gel patch, which is more like a matrix patch. In some embodiments, agents for increasing the tackiness of the gelled formulation can also be added. These agents include, but not limited to, polyvinyl pyrrolidone, acrylic polymers, or their derivatives. Still further, if a semi-permeable membrane is positioned to contact the skin surface of a subject such that the drug-containing gelled emulsion passes the drug through the semi-permeable membrane; the drug delivery system can be a reservoir patch. Alternatively, the drug delivery system can be in the form of a topical lotion or cream, for example.

As described, the drug-containing oil phase of the gelled emulsion or the microemulsion embodiments typically includes a non-gellable hydrophobic solvent. Examples of non-gellable hydrophobic solvents are essential oils, vegetable oils, and animal fat oils. More specifically, non-gellable hydrophobic solvents that can be used with certain drugs include eugenol, rose oil, wintergreen oil, eucalyptus oil, Vitamin E or its derivatives, caster oil, soy bean oil, oleic acid or its derivatives, ethyl oleate, glycerol monolaurate, and propylene glycol monolaurate, and mixtures thereof.

Drugs that can be utilized in systems in accordance with embodiments of the present invention include many lipophilic drugs. More specifically, benzodiazepines, steroids, anti-emetics, local anesthetics, antibiotics, analgesics, antiemetics, anti-inflammatory agents, nicotine, anti-migraine agents, anti-hypertension agents, hormones and retinoids can be used.

In a related detailed embodiment, a gel patch for dermal drug delivery can comprise an impermeable backing film, a drug-containing gelled emulsion or gelled microemulsion, and an adhesive. The drug-containing gelled emulsion can be in contact with the backing film and also be configured to directly contact a skin surface of a subject. The drug-containing gelled emulsion can include a continuous gelled aqueous phase and a discontinuous drug-containing oil phase dispersed within the continuous gelled aqueous phase. The drug-containing oil phase can comprise a pharmaceutically active lipophilic drug and an oil, wherein the lipophilic drug is at least five times more soluble in the oil than in water. The adhesive can be on the backing film and positioned peripherally with respect to the drug-containing gelled emulsion. The adhesive can further be configured to adhere the backing film to the skin surface, thus substantially sealing the drug-containing gelled emulsion between the skin surface and the backing film.

Turning to several specific formulations in accordance with embodiments of the present invention, the following preparative scheme can be carried out. An aqueous phase can be prepared as follows, (a) dissolve 20 wt % polyvinyl alcohol (PVA, gelling agent) in water and (b) dissolve 0.4% Pemulen TR2 (Acrylates/C10-30 alkyl acrylate crosspolymer, emulsifying agent, from Noveon, Inc., Cleveland, Ohio) in water. Mix the two aqueous solutions at about a 1:1 weight ratio until thoroughly mixed. An oil phase can be prepared by dissolving an excess amount of alprazolam into eugenol. The oil phase with the drug present can then be added to the aqueous phase, and then the two phases can then be agitated to form an emulsion. Though the emulsifying agent is present in the aqueous phase, it can likewise or alternatively be included in the oil phase. Alternatively, the emulsifying agent can be admixed therein when the oil phase and the aqueous phase are combined. The emulsion, once formed, can then be cast onto a fabric material impregnated with sodium borate, which permeates into the cast emulsion layer and acts to gel the aqueous phase by causing a crosslinking reaction with the polyvinyl alcohol. Since the aqueous phase is the continuous phase (and the oil phase is the discontinuous phase), in the emulsion formulation, the gelling of the aqueous phase solidifies the entire formulation into a soft solid and coherent layer. In this state, the composition can be applied to the skin for delivery of the benzodiazepine active agent, such as in a matrix dermal delivery patch.

To illustrate another specific embodiment, the following preparative scheme can be carried out. An aqueous phase can be prepared by dissolving 20 wt % polyvinyl alcohol (PVA, gelling agent) in water. An oil phase can be prepared by dissolving excess alprazolam into isopropyl myristrate (oil phase). The oil phase with a saturated amount of drug present can then be added to the aqueous phase. Next, Tween 80/ethanol solution can then be added in a drop wise fashion until a clear emulsion is formed. The microemulsion, once formed, can then be cast onto a fabric material impregnated with sodium borate, which permeates into the cast emulsion layer and acts to gel the aqueous phase by causing a crosslinking reaction with the polyvinyl alcohol. Since the aqueous phase is the continuous phase (and the oil phase is the discontinuous phase), in the microemulsion formulation, the gelling of the aqueous phase solidifies the entire formulation into a soft solid and coherent layer. In this state, the composition can be applied to the skin for delivery of the benzodiazepine active agent, such as in a matrix or gel patch dermal delivery system.

To illustrate still another embodiment in accordance with embodiments of the present invention, the following preparative scheme can be carried out. A microemulsion can be prepared by adding excess amount of alprazolam or another lipophilic drug into an oil phase (oleyl alcohol). The oil phase with saturated amount of drug can then be added to a fixed amount of a 50% ethanol in water solution, followed by the drop wise addition of Tween 80 until a microemulsion is formed. This microemulsion is then incorporated into a 20% PVA in water solution, the addition of the microemulsion results in the formation of a cloudy emulsion. The emulsion, once formed, can then be cast onto a fabric material impregnated with sodium borate, which permeates into the cast emulsion layer and acts to gel the aqueous phase by causing a crosslinking reaction with the polyvinyl alcohol. Since the aqueous phase is the continuous phase (and the oil phase is the discontinuous phase), in the emulsion formulation, the gelling of the aqueous phase solidifies the formulation as a whole into a soft solid and coherent layer. In this state, the composition can be applied to the skin for delivery of the benzodiazepine active agent, such as in a matrix or gel patch dermal delivery system.

In accordance with these and other manufacturing methods, in one embodiment, the emulsion can be gelled by a crosslinking process within 30 minutes. Once the oil phase and the aqueous phase are emulsified, and once the aqueous phase is gelled, the emulsion will remain as formed due to the solid characteristics of the gelled aqueous phase. As such, the use of an emulsifying agent is not strictly required, provided the aqueous phase can be sufficiently gelled before phase separation can occur. The slowing of phase separation may be helped by increasing the viscosity of the aqueous phase by adding viscosity increasing agents. By removing the requirement of the use of an emulsifying agent, more freedom in selecting other ingredients of the formulations can be realized. Further, though gelling is described as one means of solidifying the emulsion, other techniques can be used, including the use of freeze-thaw cycles, e.g., using polyvinyl alcohol, or radiation, e.g., using polyvinyl pyrrolidone.

In another embodiment, the aqueous phase of the drug-containing emulsion can include water, a gel-forming component, and a gel-triggering agent. The gel-forming component(s) can include gel forming monomers, such as vinyl alcohols, N-vinyl pyrrolidones, and sulphonated compounds such as 2-acrylamido 2-methyl 1-propane sulfonic acid (AMPS); and the gel triggering agent can be a photoiniator such as hydroxyl cyclohexyl phenyl ketone (Irgacure 184). Typically, the gel formulation is prepared by applying UV light (at a wavelength range from 240 to 420 nm) after it has been spread or coated as a layer on a release liner or other solid substrate.

The aqueous phase of the oil-in-water emulsion can also be gelled by using a gelling agent in the aqueous phase to form a thermo-reversible gel. Such a gel can be configured to liquefy when heated and re-solidify after cooling. For example, using carrageenin as a gelling agent in water can produce a gel that melts when heated, i.e. above 60 to 80° C., and solidify when it is cooled. A heated or melted form of such an emulsion formulation can be fluid and can be cast into a thin layer. Cooling of such a layer can solidify the formulation.

Thus, in an alternative embodiment, the drug-containing gelled emulsion can be formed using compositions that do not require a gel-triggering agent. For example, a composition that can be a gel at room temperature or body temperature can be heated to form a liquid. Once in a liquid state, the discontinuous drug-containing oil phase can be dispersed in the liquid and the composition as a whole can be cooled to form the drug-containing gelled emulsion. Carrageenan, pectin, and gelatin are examples of a material that can be used as the aqueous phase gel.

Typically, the drug-containing oil phase includes a lipophilic drug and a non-gellable hydrophobic solvent (non-gellable oil) selected to dissolve the drug chosen for delivery. As the hydrophobic solvent is typically non-gellable, emulsifying the material in an aqueous phase, and gelling the aqueous phase provides acceptable drug solubility as well as a stable system that can be incorporated in a dermal patch. Examples of non-gellable hydrophobic solvents include eugenol, rose oil, eucalyptus oil, other essential oils, oleyl alcohol, octyldodecanol, oleic acid, and methyl salicylate. Examples of lipophilic drugs that can be used include benzodiazepines, steroids, local anesthetics, antiememtics, anti-inflammatory agents, antibiotics, and retinoids.

Dermal Delivery Systems

In accordance with the above embodiments, the formulations of the present invention can be gelled emulsion formulations, microemulsions formulations, or gelled microemulsion formulations. When the formulation is a gelled formulation, the composition can be present as part of i) a reservoir patch, or ii) a gel patch. However, when the formulation is not a gelled formulation, then the composition can be present in a i) reservoir patch, or ii) a topical lotion, ointment, or cream.

A gel patch dermal delivery system having an impermeable backing that carries a gelled emulsion can be used to favorably exemplify embodiments of the present invention. In this embodiment, the gelled emulsion is contacted directly to a skin surface of a subject, and the lipophilic drug contained in the gelled emulsion is transported through the skin surface. In one embodiment, the drug can form a drug depot that collects and passes drug to and from beneath the skin surface, such as within the epidermis, dermis, and/or subcutaneous layer, though this is not required. In the drug depot embodiments, the drug can enter the systemic circulation directly and establish a baseline plasma concentration, and at the same time, the drug can form a depot beneath the skin surface. Once the depot is formed, the level of drug can be increased systemically by applying heat to the skin above the depot, thereby rapidly dumping drug from the depot into systemic circulation. Though this embodiment describes the concept of depot formation and baseline drug delivery, with increased drug administration systemically upon the application of heat, the transdermal patch formulations of the present invention do not necessarily have to deliver drug by this method.

### EXAMPLES

The following example illustrates the embodiments of the invention that are presently best known. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following example provides further detail in connection with what is presently deemed to be the most practical and preferred embodiments of the invention.

### Example 1

### Skin Permeation Methodology

In order to assess the influence of solvents on skin permeability of alprazolam, in vitro skin flux of alprazolam in various liquid formulations was tested. All liquid formulations contained excess alprazolam in solution. In the study, hairless mouse skin (HMS) was used for the in vitro testing. Freshly separated epidermis removed from the abdomen was mounted carefully between two cells of a Franz diffusion cell. The receiver chamber of the cell was filled with pH 7.4 phosphate buffered saline (PBS). The experiment was initiated by placing the test formulation on the stratum corneum (SC). Franz cells were placed in a Franz diffusion cell console (Logan Instruments Corp. Model #: FDC-24) maintained at 37° C. At predetermined time intervals, an 800 μL aliquot was withdrawn and replaced with fresh PBS solution. Skin flux (μg/cm²/h) was determined from the steady-state slope of a plot of the cumulative amount of benzodiazepine that permeates versus time.

The steady-state flux of alprazolam from the test formulations through HMS maintained at 37° C. is presented in Table 1 below.

TABLE 1

| | Skin Flux |
|---|---|
| Formulation | (μg/cm²/h) |
| PBS alone | 0.1 ± 0.07 |
| 20% ethanol | 1.0 ± 0.6 |
| 80% PBS | |
| 40% ethanol | 4 ± 1 |
| 60% PBS | |
| 25% IPM | 3 ± 1 |
| 30% Ethanol:water (1:1) | |
| 45% Tween 80 | |
| (microemulsion) | |
| 100% Eugenol | 4 ± 1 |
| 100% poly ethylene glycol | 0.2 ± 0.08 |
| 400 (PEG 400) | |

As can be seen in Table 1, simply putting alprazolam in an aqueous solution (PBS) or in PEG 400 resulted in formulations that produced far from sufficient flux, assuming 0.5 mg to 7 mg alprazolam per day can be used to effectively treat panic disorder. The use of ethanol, which has excellent solvent properties for alprazolam, significantly increases the flux. The microemulsion formulation also produced adequate flux.

### Example 2

### In Vitro Skin Flux of Alprazolam from PVA Hydrogels

Several polyvinyl alcohol hydrogel formulations with excess alprazolam were prepared as follows:

Formulation 1

Part A: 5 wt % eugenol in water emulsion, 0.4 wt % TR-2 emulsifier, and excess amount of alprazolam.

Part B: 17 wt % polyvinyl alcohol in water.

Formulation 1 was obtained by aggressively mixing one weight portion of Part A with one weight portion of Part B.

Formulation 2

Part A: 10 wt % eugenol in water emulsion, 0.4 wt % TR-2 emulsifier, and excess amount of alprazolam.

Part B: 17 wt % polyvinyl alcohol in water.

Formulation 2 was obtained by aggressively mixing one weight portion of Part A with one weight portion of Part B.

Formulation 3

Part A: emulation of 34 wt % IPM (isopropyl myristate), 24 wt % ethanol, 24 wt % water, 18 wt % Tween 80, and excess amount of alprazolam.

Part B: 17 wt % polyvinyl alcohol in water.

Formulation 3 was obtained by aggressively mixing one weight portion of Part A with one weight portion of Part B.

Formulation 4

6 wt % PVA, 34 wt % water, 60 wt % N-methyl pyrrolidone (NMP), and excess amount of alprazolam.

Formulation 5

13.5 wt % polyvinyl alcohol, 77.5 wt % water, 10 wt % N-methyl pyrrolidone (NMP), and excess amount of alprazolam.

Formulation 6

28% polyvinyl alcohol, 65% water, 7% rose oil, and excess alprazolam.

Each of the six viscous solutions were disposed on to 25 cm² piece of Dexter non-woven material that was pretreated with 1 mL of a 2 wt % sodium borate solution. In each case, the solutions formed a solidified gel within approximately 30 minutes. Each of the gelled formulations was cut into a 2 cm² piece and placed on a stratum corneum (SC) and mounted on a diffusion cell for flux measurements, as described in Example 1. The results of these tests are presented in Table 2 below.

TABLE 2

| | Ingredients in addition to | |
|---|---|---|
| | excess amount of | Skin Flux |
| Formulation | alprazolam | (μg/cm2/h) |
| 1 | 2.5% Eugenol | 1.3 ± 1.1 |
| | 0.2% TR-2 | |
| | 8.5% PVA in water | |
| 2 | 5% Eugenol | 5 ± 1 |
| | 0.2% TR-2 | |
| | 8.5% PVA in water | |
| 3 | 17% IPM | 0.5 ± 0.2 |
| | 12% ethanol | |
| | 9% Tween 80 | |
| | 8.5% PVA | |
| | 53.5% water | |
| 4 | 6% PVA | 1.1 ± 0.1 |
| | 60% NMP | |
| | 34% water | |
| 5 | 13.5% PVA | 0.3 ± 0.07 |
| | 10% NMP | |
| | 76.5% water | |
| 6 | 28% PVA | 3 ± 1 |
| | 7% Rose Oil | |
| | 65% water | |

As can be seen in Table 2, even relatively small amounts of eugenol or rose oil in the formulation produced an increased flux. Each of the formulations described in the present example can be gelled into a thin layer for incorporation into a gel patch in accordance with embodiments of the present invention.

### Example 3

### Gelled Emulsion Formulations

Prototype gellable emulsion and microemulsion formulations were prepared by mixing the following formulation components according to Table 3, as follows:

TABLE 3

| Type of gellable formulation | Ingredients |
|---|---|
| Emulsion | 3% oleyl alcohol |
| | 0.4% TR-2 |
| | 87% water |
| | 9.6% PVA |
| Microemulsion 1 | 22% ethanol |
| | 17% Tween 80 |
| | 6% oleyl alcohol |
| | 10% PVA |
| | 55% water |
| Microemulsion 2 | 6% octyl dodecanol |
| | 20% ethanol |
| | 24% Tween 80 |
| | 10% PVA |
| | 40% water |
| Microemulsion 3 | 6% oleyl alcohol |
| | 13% Tween 80 |
| | 24% Labrasol |
| | 11% PVA |
| | 46% water |

A layer of each formulation was cast on a fabric material impregnated with sodium borate, and in each case, the aqueous phase solidified into a soft solid.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

1. A drug-containing gelled emulsion, comprising:
a) a continuous gelled aqueous phase; and
b) a discontinuous drug-containing oil phase dispersed within the continuous gelled aqueous phase, said oil phase comprising a pharmaceutcally active lipophilic drug and an oil, said lipophilic drug being at least five times more soluble in said oil than in water, and said drug-containing gelled emulsion being present in a dermal delivery system.

2. A drug-containing gelled emulsion as in claim 1, wherein the lipophilic drug is at least twenty times more soluble in said oil than in water.

3. A drug-containing gelled emulsion as in claim 1, wherein the lipophilic drug is at least one hundred times more soluble in said oil than in water.

4. A drug-containing gelled emulsion as in claim 1, wherein the dermal delivery system is a transdermal delivery patch.

5. A drug-containing gelled emulsion as in claim 1, wherein the continuous gelled aqueous phase includes water and a gel-forming component.

6. A drug-containing gelled emulsion as in claim 2, wherein the aqueous phase further comprises a gel triggering agent.

7. A drug-containing gelled emulsion as in claim 2, wherein the gel-forming component is a gel-forming polymer.

8. A drug-containing gelled emulsion as in claim 7, wherein the gel-forming polymer is polyvinyl alcohol.

9. A drug-containing gelled emulsion as in claim 6, wherein the gel-triggering agent is boric acid or a salt of boric acid.

10. A drug-containing gelled emulsion as in claim 1, wherein the continuous gelled aqueous phase comprises a thermal gel that is flowable when heated above its melting point, wherein the discontinuous drug-containing oil phase is dispersed in the thermal gel above the melting point, and wherein the drug-containing gelled emulsion is formed upon cooling the thermal gel below the melting point.

11. A drug-containing gelled emulsion as in claim 10, wherein the thermal gel comprises one or more gel-forming agent selected from the group consisting of carrageenan, pectin, and gelatin.

12. A drug-containing gelled emulsion as in claim 1, wherein the drug-containing oil phase includes a nongellable oil.

13. A drug-containing gelled emulsion as in claim 12, wherein the non-gellable oil is selected from the group consisting of essential oils, vegetable oils, and animal fat oils.

14. A drug-containing gelled emulsion as in claim 12, wherein the non-gellable hydrophobic solvent is selected from the group consisting of eugenol, rose oil, wintergreen oil, eucalyptus oil, Vitamin E or Its derivatives, caster oil, soy bean oil, oleic acid or its derivatives, ethyl oleate, glycerol monolaurate, propylene glycol monolaurate, and mixtures thereof.

15. A drug-containing gelled emulsion as in claim 1, wherein the lipophilic drug is selected from the group consisting of benzodiazepines, steroids, anti-emetics, local anesthetics, antibiotics, analgesics, antiemetics, anti-inflammatory agents, nicotine, anti-migraine agents, anti-hypertension agents, hormones, and retinoids.

16. A drug-containing gelled emulsion as in claim 1, wherein the lipophilic drug is alprazolam.

17. A method of preparing a drug-containing gelled emulsion for dermal drug delivery, comprising:
a) forming a drug-containing oil phase comprising a lipophilic drug and an oil, said lipophilic drug being at least five times more soluble in said oil than in water;
b) forming an aqueous phase comprising water and a gel-forming component;
c) emulsifying the oil phase with the aqueous phase to form an oil-in-water-emulsion;
d) incorporating the oil-in-water emulsion in a dermal delivery system; and
e) gelling the hydrophilic phase before or after incorporating the oil-in-water dispersed emulsion in a dermal delivery system.

18. A method as in claim 17, wherein the lipophilic drug is at least twenty times more soluble in said oil than in water.

19. A method as in claim 17, wherein the lipophilic drug is at least one hundred times more soluble in said oil than in water.

20. A method as in claim 17, wherein the dermal delivery system is a transdermal delivery patch.

21. A method as in claim 17, wherein the gelling is started by contacting the aqueous phase with a gel-triggering agent.

22. A method as in claim 17, wherein the gel-forming component is polyvinyl alcohol.

23. A method as in claim 21, wherein the gel-triggering agent is boric acid or a salt of boric acid.

24. A method as in claim 17, wherein the emulsifying step occurs by heating the aqueous phase to form a liquid state and forming the oil-in-water emulsion while the aqueous phase is in the liquid state, and the gelling step occurs by cooling the oil-dispersed emulsion.

25. A method as in claim 17, wherein the drug-containing oil phase includes a non-gellable oil.

26. A method as in claim 25, wherein the non-gellable hydrophobic solvent is selected from the group consisting of essential oils, vegetable oils, and animal fat oils.

27. A method as in claim 25, wherein the non-gellable hydrophobic solvent is selected from the group consisting of eugenol, rose oil, wintergreen oil, eucalyptus oil, Vitamin E or its derivatives, caster oil, soy bean oil, oleic acid or its derivatives, ethyl oleate, glycerol monolaurate, propylene glycol monolaurate, and mixtures thereof.

28. A method as in claim 17, wherein the lipophilic drug is selected from the group consisting of benzodiazepines, steroids, anti-emetics, local anesthetics, antibiotics, analgesics, antiemetics, anti-inflammatory agents, nicotine, anti-migraine agents, anti-hypertension agents, hormones, and retinoids.

29. A method as in claim 17, wherein the lipophilic drug is alprazolam.

30. A drug-containing microemulsion, comprising:
a) a continuous aqueous phase;
b) a discontinuous drug-containing oil phase comprising a lipophilic drug and an oil, said lipophilic drug being at least five times more soluble In said oil than in water, and said discontinuous oil phase dispersed in the continuous aqueous phase to form an oil-in-water microemulsion;
c) at least one surfactant substantially positioned interfacially between the continuous aqueous phase and the discontinuous oil phase, said drug-containing microemulsion being present in a dermal delivery system.

31. A drug-containing microemulsion as in claim 30, wherein the lipophilic drug is at least twenty times more soluble in said oil than in water.

32. A drug-containing microemulsion as in claim 30, wherein the lipophilic drug is at least one hundred times more soluble in said oil than in water.

33. A drug-containing microemulsion as in claim 30, wherein the dermal delivery system is a transdermal delivery patch.

34. A drug-containing microemulsion as in claim 30, wherein the microemulsion includes water and at least one surfactant consisting of Pemulen TR-2, fatty alcohols, Mono- and diglycerides, and mixtures thereof.

35. A drug-containing microemulsion as in claim 30, wherein the surfactant is selected from the group consisting of nonionic surfactants and zwitterionic surfactants.

36. A drug-containing microemulsion as in claim 30, wherein the surfactant is selected from the group consisting of short chain alcohols, plurol isostearique, Tweens, Spans, Chemophor RH, soybean lecithin, Labrasol Pemulen TR-2, fatty alcohols, monoglycerides, dilycerides, and mixtures thereof.

37. A drug-containing microemulsion as in claim 30, wherein the lipophilic drug is selected from the group consisting of benzodiazepines, steroids, anti-emetics, local anesthetics, antibiotics, analgesics, antiemetics, anti-inflammatory agents, nicotine, anti-migraine agents, anti-hypertension agents, hormones, and retinoids.

38. A drug containing microemulsion as In claim 30, wherein the discontinuous oil phase includes a hydrophobic solvent selected from the group consisting of essential oils, vegetable oils, and animal fat oils.

39. A drug-containing microemulsion as in claim 30, wherein the discontinuous oil phase includes a hydrophobic solvent selected from the group consisting of eugenol, rose oil, wintergreen oil, eucalyptus oil, Vitamin E or its derivatives, caster oil, soy bean oil, oleic acid or its derivatives, ethyl oleate, glycerol monolaurate, propylene glycol monolaurate, and mixtures thereof.

40. A drug-containing microemulsion as in claim 30, wherein the microemulsion has the appearance of a clear solution.

41. A drug-containing microemulsion as in claim 30, wherein the continuous aqueous phase is present at from 5 wt % to 95 wt %.

42. A drug-containing microemulsion as in claim 30, wherein the discontinuous oil phase is present at from about 0.1 wt % to 30 wt %.

43. A drug-containing microemulsion as in claim 30, wherein the at least one surfactant is present at from 0.1 wt % to 95 wt %.

44. A drug-containing microemulsion as in claim 30, wherein multiple co-surfactants are present.

45. A drug-containing microemulsion as in claim 30, wherein the discontinuous oil phase includes aggregates with an average size less than about 400 nm.

46. A drug-containing microemulsion as in claim 30, wherein the continuous aqueous phase is gelled.

47. A method of preparing a drug-containing microemulsion for dermal delivery, comprising:
a) forming a drug-containing oil phase comprising a lipophilic drug and an oil, said lipophilic drug being at least five times more soluble in said oil than in water;
b) forming an aqueous phase;
c) emulsifying the aqueous phase with the oil phase in the presence of at least one surfactant to form the drug-containing microemulsion, wherein the surfactant is substantially positioned interfacially between a continuous aqueous phase and a dispersed discontinuous oil phase; and
d) incorporating the drug-containing microemulsion in a dermal delivery system.

48. A method as in claim 47, wherein the lipophilic drug is at least twenty times more soluble in said oil than in water.

49. A method as in claim 47, wherein the lipophilic drug is at least one hundred times more soluble in said oil than in water.

50. A method as in claim 47, wherein the dermal delivery system is a transdermal delivery patch.

51. A method as in claim 47, wherein the surfactant is selected from the group consisting of nonionic surfactants and zwitterionic surfactants.

52. A method as in claim 47, wherein the at least one surfactant is selected from the group consisting of short chain alcohols, plurol isostearique, Tweens, Span 20, Chemophor RH, soybean lecithin, Labrasol, Pemulen TR-2, fatty alcohols, monoglycerides, dilycerides, and mixtures thereof.

53. A method as in claim 47, wherein the lipophilic drug is selected from the group consisting of benzodiazepines, steroids, anti-emetics, local anesthetics, antibiotics, analgesics, antiemetics, anti-inflammatory agents, nicotine, anti-migraine agents, anti-hypertension agents, hormones, and retinoids.

54. A method as in claim 47, wherein the discontinuous oil phase includes a hydrophobic solvent selected from the group consisting of essential oils, vegetable oils, and animal fat oils.

55. A method as in claim 47, wherein the discontinuous oil phase includes a hydrophobic solvent selected from the group consisting of eugenol, rose oil, wintergreen oil, eucalyptus oil, Vitamin E or its derivatives, caster oil, soy bean oil, oleic acid or its derivatives, ethyl oleate, glycerol monolaurate, propylene glycol monolaurate, and mixtures thereof.

56. A method as in claim 47, wherein the microemulsion has the appearance of a clear solution.

57. A method as in claim 47, wherein the aqueous phase is present at from 5 wt % to 95 wt %.

58. A method as in claim 47, wherein the discontinuous oil phase is present at from 0.1 wt % to 30 wt %.

59. A method as in claim 47, wherein the at least one surfactant is present at from 0.1 wt % to 95 wt %.

60. A method as in claim 47, wherein the discontinuous oil phase includes aggregates with an average size less than about 400 nm.

62. A gel patch for transdermal drug delivery, comprising:
a) an impermeable backing film;
b) a drug-containing gelled emulsion being in contact with the backing film and also being configured to directly contact a skin surface of a subject, said drug-containing gelled emulsion including:
i) a continuous gelled aqueous phase, and
ii) a discontinuous drug-containing oil phase dispersed within the continuous gelled aqueous phase, said oil phase including a lipophilic drug and an oil, said drug being at least five times more soluble in said oil than in water; and
c) an adhesive on the backing film positioned peripherally with respect to the drug-containing gelled emulsion, said adhesive being configured to adhere the backing film to the skin surface, thus substantially sealing the drug-containing gelled emulsion within an enclosure defined by the skin surface and the backing film.

63. A gel patch for transdermal drug delivery as in claim 62, wherein the drug-containing gelled emulsions further comprises a surfactant, and wherein the emulsion is a microemulsion.
